(19) United States
(12) Patent Application Publication
(10) Pub. No.: US 2022/0111142 A1
(43) Pub. Date: Apr. 14, 2022

(54) MEDICAL INFUSION PUMP HOLDER

(71) Applicant: Michael Senerchia, Bayonne, NJ (US)

(72) Inventor: Michael Senerchia, Bayonne, NJ (US)

(21) Appl. No.: 17/556,517

(22) Filed: Dec. 20, 2021

Related U.S. Application Data
(63) Continuation-in-part of application No. 16/424,069, filed on May 28, 2019.

(51) Int. Cl. A61M 5/142

(52) U.S. Cl. CPC A61M 5/14244; A61M 2005/1586

(57) ABSTRACT

The present invention relates to a medical infusion pump holder wearable as a glove, band, or sleeve that fits over the hand, arm, or leg of a patient and holds a medical infusion pump stationary proximate an injection site on the patient while the patient's arms or legs are in motion. The medical infusion pump holder comprises a receptacle that is formed from a flexible mesh material having a plurality of holes dispersed throughout. The medical infusion pump is inserted into the receptacle with only the intravenous drug delivery tube egress from the receptacle. A bottom seal is integrated around the periphery of the bottom end and closes the bottom end, and a top seal is fastened around the periphery of the top end. The top seal is stretchable and under a no-load condition draws the top end substantially closed retaining the medical infusion pump inside the receptacle.

### CROSS-REFERENCE TO RELATED APPLICATIONS

This application contains subject matter which is related to the subject matter of the following co-pending application. The below-listed application is hereby incorporated herein by reference in its entirety:

This is a U.S. non-provisional application that is a continuation in part of a U.S. non-provisional application Ser. No. 16/424,069, inventor Michael Senerchia, entitled “MEDICAL INFUSION HOLDER”, filed May 28, 2019.

### TECHNICAL FIELD OF THE INVENTION

This invention relates to a medical infusion pump holder wearable as a glove, band, or sleeve that fits over the hand, arm, or leg of a patient and holds a medical infusion pump stationary proximate an injection site on a patient while the patient's arms or legs are in motion.

### BACKGROUND OF THE INVENTION

Before our invention patients would be required to sit stationary for several hours to receive a drug infusion intravenously. An infusion pump tethered with drug delivery tubes would be mounted on a tripod pole or otherwise separated from the patient. This infusion pump separation from the patient and tethering with tubes while drugs are being delivered at an injection site on the patient had the effect of restricting the patient's motion particularly the arms, hands, or legs proximate to the injection site.

An additional shortcoming of the prior art is that often taking several hours to deliver the drugs to the patient much time and disruption to the patient's daily routines occurred while receiving the drug infusion.

The present invention addresses these and other shortcomings by providing a medical infusion pump holder. For these reasons and shortcomings as well as other reasons and shortcomings there is a long-felt need that gives rise to the present invention.

### SUMMARY OF THE INVENTION

The shortcomings of the prior art are overcome and additional advantages are provided through the provision of a medical infusion pump holder comprising a receptacle formed from a flexible mesh material having a plurality of holes dispersed throughout. The receptacle has a bottom end and a top end. A medical infusion pump is inserted into the receptacle. The receptacle applies uniform constricting pressure across the surface of the medical infusion pump to secure the medical infusion pump from movement during motion by a patient. An intravenous drug delivery tube is detachably connected to the medical infusion pump. Only the intravenous drug delivery tube egresses from the receptacle. A bottom seal is integrated around the periphery of the bottom end and closes the bottom end. A top seal is fastened around the periphery of the top end. The top seal is stretchable and under a no-load condition draws the top end substantially closed retaining the medical infusion pump inside the receptacle. A restraint band forms a continuous loop, is stretchable, and is fastened to the receptacle proximate to the bottom end. A thumb band forms a continuous loop, is stretchable, and is fastened to the restraint band, and a stabilizing band has a resealable closure, is non-stretchable, and is fastened proximate to the top end. Wherein the medical infusion pump holder is configured to slide over the hand of a patient. The patient's thumb slides into the thumb band preventing the medical infusion pump holder from rotating around the hand of the patient. The restraint band wraps around the hand of the patient, and the stabilizing band is tightened and secured by the patient around the patient's forearm.

Additional shortcomings of the prior art are overcome and additional advantages are provided through the provision of a medical infusion pump holder comprising a receptacle formed from a flexible mesh material having a plurality of holes dispersed throughout. The receptacle has a bottom end and a top end. A medical infusion pump is inserted into the receptacle. The receptacle applies uniform constricting pressure across the surface of the medical infusion pump to secure the medical infusion pump from movement during motion by a patient. An intravenous drug delivery tube is detachably connected to the medical infusion pump. Only the intravenous drug delivery tube egresses from the receptacle. A bottom seal is integrated around the periphery of the bottom end and closes the bottom end. A top seal is integrated around the periphery of the top end. The top seal is stretchable and under a no-load condition draws the top end substantially closed retaining the medical infusion pump inside the receptacle. A circular hand wrap has a finger opening, a wrist opening, a thumb opening, and a top side, the receptacle is fastened to the top side fitting between the finger opening and the wrist opening with the bottom seal facing the finger opening and the top seal facing the wrist opening. A wrist seal is fastened around the periphery of the wrist opening, the wrist seal is stretchable and under a no-load condition draws the wrist opening substantially closed. Wherein the medical infusion pump holder is configured to receive the hand of a patient in a loose-fitting manner minimizing sensitive skin irritation. The patient's thumb slides into the thumb opening preventing the medical infusion pump holder from rotating around the hand of the patient. The circular hand wrap substantially encloses the top portion and palm of the patient hand.

Additional shortcomings of the prior art are overcome and additional advantages are provided through the provision of a medical infusion pump holder comprising a receptacle formed from a flexible mesh material having a plurality of holes dispersed throughout. The receptacle has a bottom end and a top end. A medical infusion pump is inserted into the receptacle. The receptacle applies uniform constricting pressure across the surface of the medical infusion pump to secure the medical infusion pump from movement during motion by a patient. An intravenous drug delivery tube is detachably connected to the medical infusion pump. Only the intravenous drug delivery tube egresses from the receptacle. A bottom seal is integrated around the periphery of the bottom end and closes the bottom end. A top seal is integrated around the periphery of the top end. The top seal is stretchable and under a no-load condition draws the top end substantially closed retaining the medical infusion pump inside the receptacle. A circular sleeve formed of a compression non-slip material having a first edge, a second edge, and a top side, the receptacle is fastened to the top side fitting between the first edge and the second edge with the bottom end facing the first edge and the top end facing the second edge. Wherein the medical infusion pump holder is configured to receive the arm or leg of a patient through the circular sleeve.

Additional shortcomings of the prior art are overcome and additional advantages are provided through the provision of a method of using a medical infusion pump holder comprising fitting a medical infusion pump holder over the arm, leg, or hand of a patient.

The method continues by inserting a medical infusion pump in a receptacle, the receptacle is formed from a flexible mesh material having a plurality of holes dispersed throughout. The receptacle has a bottom end and a top end. The medical infusion pump is inserted into the receptacle with only an intravenous drug delivery tube egressing from the receptacle. A bottom seal is integrated around the periphery of the bottom end and closes the bottom end. A top seal is integrated around the periphery of the top end. The top seal is stretchable and under a no-load condition draws the top end substantially closed retaining the medical infusion pump inside the receptacle. The method then delivers a prescription drug by way of the medical infusion pump to the patient intravenously.

Additional features and advantages are realized through the techniques of the present invention. Other embodiments and aspects of the invention are described in detail herein and are considered a part of the claimed invention. For a better understanding of the invention with advantages and features, refer to the description and the drawings.

### BRIEF DESCRIPTION OF THE FIGURES

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates examples of a medical infusion pump holder;

FIGS. 2-8 illustrate one example of a medical infusion pump holder configured as a band;

FIGS. 9-15 illustrate one example of a medical infusion pump holder configured as a glove;

FIGS. 16-23 illustrate one example of a medical infusion pump holder configured as a sleeve; and

FIG. 24 illustrates one example of a method of using a medical infusion pump holder.

The detailed description explains the preferred embodiments of the invention, together with advantages and features, by way of example with reference to the drawings.

### DETAILED DESCRIPTION OF THE INVENTION

For patients that require a medical infusion of prescription drugs, the process can take several hours. During this time an intravenous (IV) tube is placed into the patient at an injection site and a medical infusion pump delivers the drugs to the patient through the IV tube. Often the patient must sit stationary while connected by IV tubes and a medical infusion pump during the treatment cycle. While sitting idle receiving the drug infusion there are a lot of activities that the patient can't simultaneously accomplish during the treatment cycle because they can not freely move their hand, arms, or legs.

An advantage, in the present invention, is that a medical infusion pump holder secures the infusion pump proximate to an injection site 306 on patient 302. This allows patient 302 to carry on with their daily activities moving their hand, arms, and legs during treatment without impairing the drug delivery process. The medical infusion pump 202, in the present invention, can be secured in place on the patient's hand, arm, or leg.

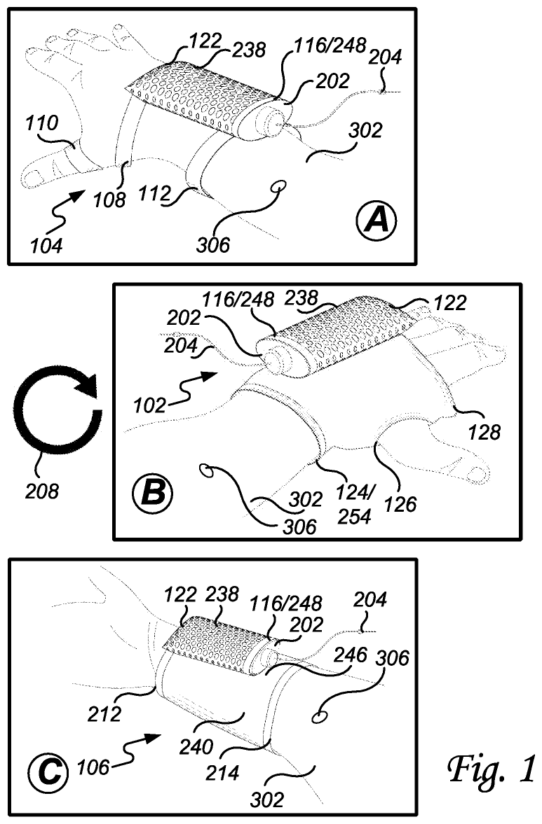

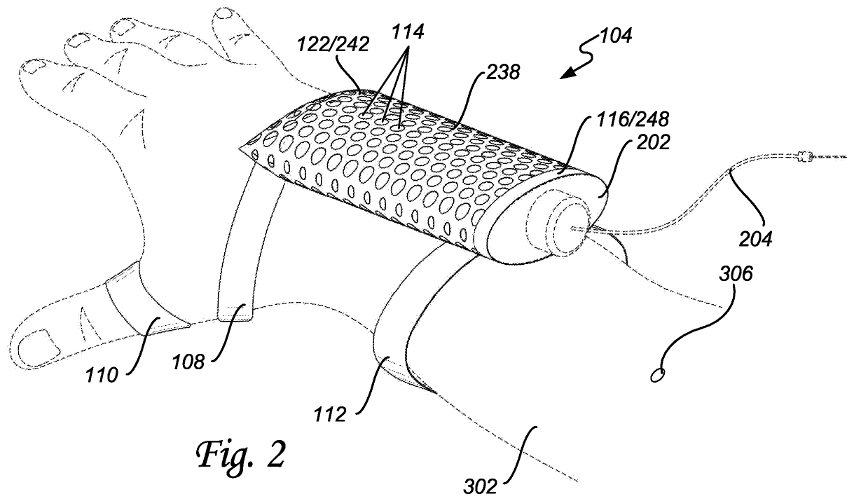

Turning now to the drawings in greater detail, it will be seen that FIG. 1 illustrates examples of a medical infusion pump holder. In common with each type of medical infusion pump holder is a receptacle 238. In an exemplary embodiment, FIG. 1, reference ‘A’, and also illustrated in at least FIG. 2, a medical infusion pump holder can be configured as a band 104 and slid over the hand of a patient 302. The patient's 302 thumb slides into the thumb band 110 preventing the medical infusion pump holder band 104 from rotating around the hand of patient 302. The restraint band 108 wraps around the hand of patient 302, and the stabilizing band 112 is tightened and secured by patient 302 around the patient's forearm. A receptacle 238 is formed from a flexible mesh material having a plurality of holes dispersed throughout. A medical infusion pump 202 is inserted into receptacle 238. The receptacle 238 applies uniform constricting pressure across the surface of the medical infusion pump 202 to secure the medical infusion pump 202 from movement during motion by the patient 302 such as arm, leg, or hand motion. An intravenous drug delivery tube 204 is detachably connected to the medical infusion pump 202. Only the intravenous drug delivery tube 204 egresses from the receptacle 238.

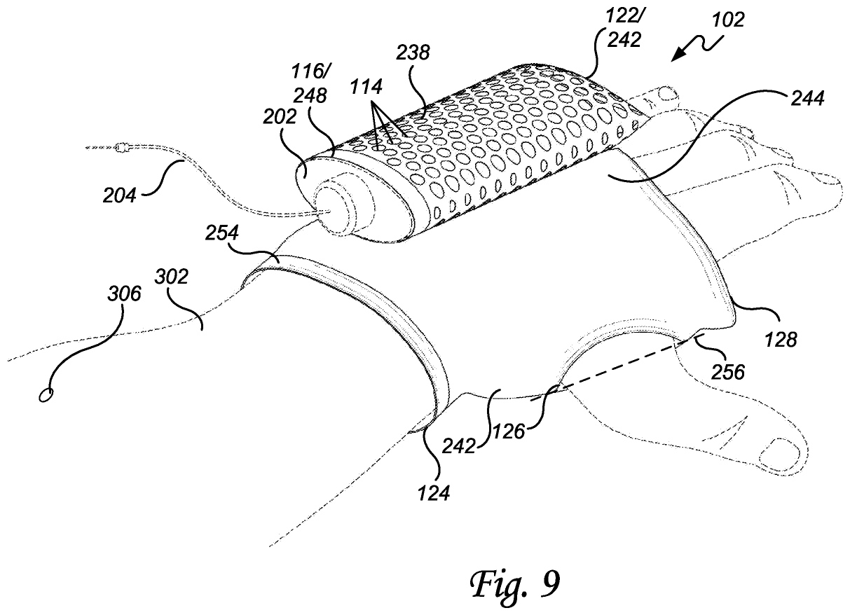

In another exemplary embodiment, FIG. 1, reference ‘B’, and also at least FIG. 9 illustrates a medical infusion pump holder configured as a glove 102 that receives the hand of a patient 302 in a loose-fitting manner minimizing sensitive skin irritation for those patients that exhibit this condition. The patient's thumb slides into the thumb opening 126 preventing the medical infusion pump holder glove 102 from rotating around the hand of the patient 302. The circular hand wrap 242 substantially encloses the top portion and palm of the patient's 302 hand. A circular hand wrap 242 has a finger opening 128, a wrist opening 124, a thumb opening 126, and a top side 244. The receptacle 238 is fastened to the top side 244 fitting between the finger opening 128 and the wrist opening 124 with the bottom end 122 facing the finger opening and the top end 116 facing the wrist opening 124.

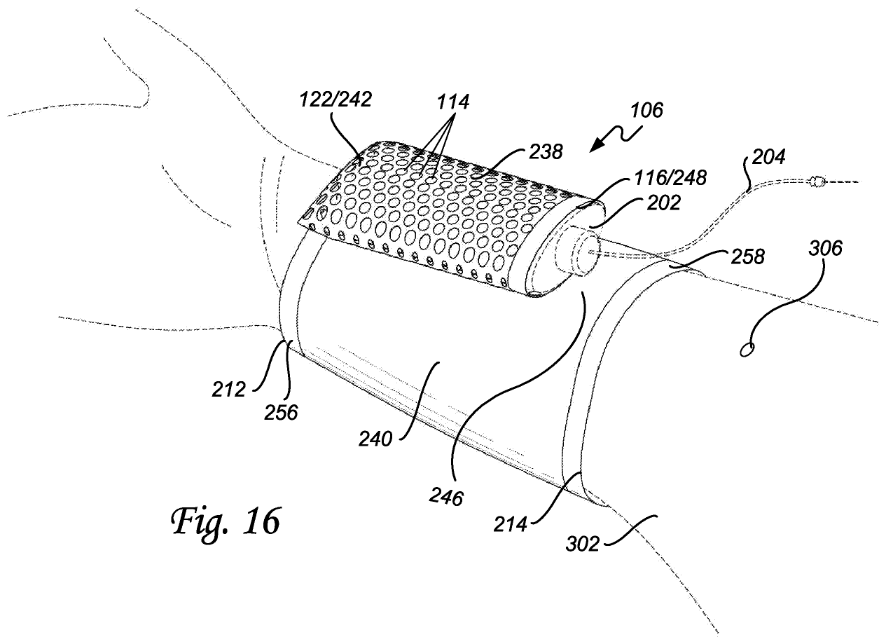

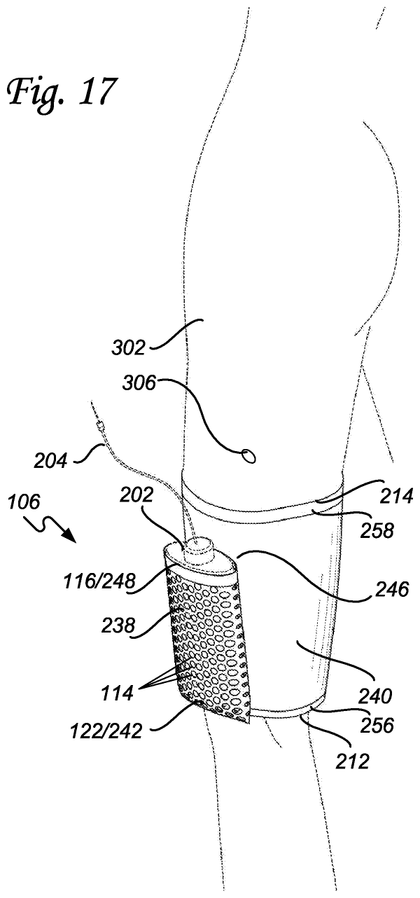

In another exemplary embodiment, FIG. 1, reference ‘C’, and also in at least FIGS. 16 and 17, there is illustrated a medical infusion pump holder configured as a sleeve 106. The medical infusion pump holder sleeve 106 receives an arm or a leg of a patient 302 through the opening in the center of the circular sleeve 240. The second edge 214 of the circular sleeve 240, has a larger diameter than the first edge 212 and as such is pulled higher on the patient's arm or leg where the diameter is greater.

In an exemplary embodiment, the circular sleeve 240 is formed of a compression non-slip material and has a first edge 212, a second edge 214, and a top side 246 the receptacle 238 is fastened to the top side 246 fitting between the first edge 212 and the second edge 214 with the bottom end 122 facing the first edge 212 and the top end 248 facing the second edge 214.

In an exemplary embodiment the medical infusion pump holder can be configured as a medical infusion pump holder band 104, a medical infusion pump holder glove 102, or a medical infusion pump holder sleeve 106. The medical infusion pump holder band 104, glove 102, and sleeve 106 can be sold individually or as a kit. In addition, the medical infusion pump holder band 104, glove 102, and sleeve 106 can be tailored in size to fit each patient's hand size, arm diameter size, or leg diameter size.

An advantage, in the present invention, is that in an exemplary embodiment, between the medical infusion pump holder band 104, glove 102, and sleeve 106 designs, the receptacle 238 can be a common shape and size and can be either configured to be removed and interchangeably used 208 with each of the medical infusion pump holder band 104, glove 102, and sleeve 106 or fastened permanently on each of the medical infusion pump holder band 104, glove 102, and sleeve 106. In this regard, receptacle 238 can be removable and interchangeably mountable to the medical infusion pump holder when the medical infusion pump holder is configured as a band 104, a glove 102, or a sleeve 106.

An advantage, in the present invention, is that in an exemplary embodiment, to control the limiting of movement of the medical infusion pump 202, receptacle 238 fits into the boundaries of the band 104, glove 102, and sleeve 106 so that none of the edges of the receptacle 238 or the medical infusion pump 202 inside the receptacle 238, except for the IV tube 204, hangs over any edge of the boundaries of the band 104, glove 102, or sleeve 106. In this regard, by securing the receptacle 238 and the medical infusion pump 202 inside the receptacle 238 within the boundaries of band 104, glove 102, and sleeve 106 the medical infusion pump 202 is precluded from shifting, bouncing, or otherwise moving the band 104, glove 102, and sleeve 106 around the patient's 302 hand, arm, or leg while the patient's 302 hand, arm, or leg is in motion.

Referring to FIG. 2, there is illustrated one example of a medical infusion pump holder configured as a band 104. In an exemplary embodiment, the medical infusion pump holder embodied as a band 104 can comprise a receptacle 238 formed from a flexible mesh material having a plurality of holes 114 dispersed throughout. The receptacle 238 has a bottom end 122 and a top end 116.

In an exemplary embodiment, a medical infusion pump 202 is inserted into receptacle 238. The receptacle 238 applies uniform constricting pressure across the surface of the medical infusion pump 202 to secure the medical infusion pump 202 from movement during motion by a patient 302 such as arm, leg, or hand motion. An intravenous drug delivery tube 204 is detachably connected to the medical infusion pump 202. Only the intravenous drug delivery tube 204 egresses from the receptacle 238.

A bottom seal 242 is integrated around the periphery of the bottom end 122 and closes the bottom end 122 at least partially. In an exemplary embodiment, the bottom seal 242 can be a zipper, safety pin, hook-and-loop such as Velcro, or other types and kinds of bottom seals that can be opened, closed, or partially closed as may be required and/or desired in a particular embodiment.

An advantage, in the present invention, is that the bottom seal can be opened, closed, or closed partially so that a tube or cord can pass out the bottom end 122 of the receptacle but the partially closed bottom seal 242 keeps the medical infusion pump 202 from sliding out the bottom end 122. In this regard, in an exemplary embodiment, in operation, the bottom seal 242 can be opened to allow cords such as a rechargeable power cord, from the back of the medical infusion pump 202 to egress the receptacle 238 while keeping the medical infusion pump 202 from sliding out of the bottom end 122 of the receptacle 238.

Alternatively, the bottom seal 242 can be a plurality of sewn stitches, adhesive, or other types and kinds of permanent bottom seals as may be required and/or desired in a particular embodiment.

A top seal 248 is fastened around the periphery of the top end 116. The top seal 248 is stretchable and under a no-load condition draws the top end 116 substantially closed retaining the medical infusion pump 202 inside the receptacle 238. In an exemplary embodiment, the top seal 248 can be an elastic band, rubber band, a drawstring, adjustable twist tie, or other types and/or kinds of seals as may be required and/or desired in a particular embodiment.

A restraint band 108 forms a continuous loop, is stretchable and is fastened to the receptacle 238 proximate to the bottom end 122. A thumb band 110 forms a continuous loop, is stretchable and is fastened to the restraint band 108. In an exemplary embodiment, the restraint band 108 and the thumb band 110 can be an elastic band or other types and kinds of stretchable bands as may be required and/or desired in a particular embodiment.

A stabilizing band 112 having a resealable closure 120, is non-stretchable, and is fastened proximate to the top end 116. In an exemplary embodiment, the stabilizing band 112 can be hook-and-loop such as Velcro that can stick to itself. The resealable closure 120 can be formed by folding backward and fastening a portion of the stabilizing band 112 onto itself forming a loop at the end of the stabilizing band 112 that becomes the resealable closure 120. In this embodiment, the resealable closure 120 can stick by way of hook-and-loop to the stabilizing band 112 in an open and close resealable manner. In this regard, the stabilizing band 112 and the resealable closure 120 can be formed from a single piece of hook-and-loop material.

An advantage, in the present invention, is that in operation, patient 302 can open the stabilizing band 112 made from a hook-and-loop material, wrap the stabilizing band 112 around their forearm, then tighten the stabilizing band 112 by closing the resealable closure 120 with a motion of pressing the resealable closure 120 against the stabilizing band 112 material.

In an exemplary embodiment, the medical infusion pump holder can be configured as a band 104 and can slide over the hand of a patient 302. The patient's 302 thumb slides into the thumb band 110 preventing the medical infusion pump holder from rotating around the hand of patient 302. The restraint band 108 wraps around the hand of patient 302. The stabilizing band 112 is tightened and secured by patient 302 around the patient's 302 forearm.

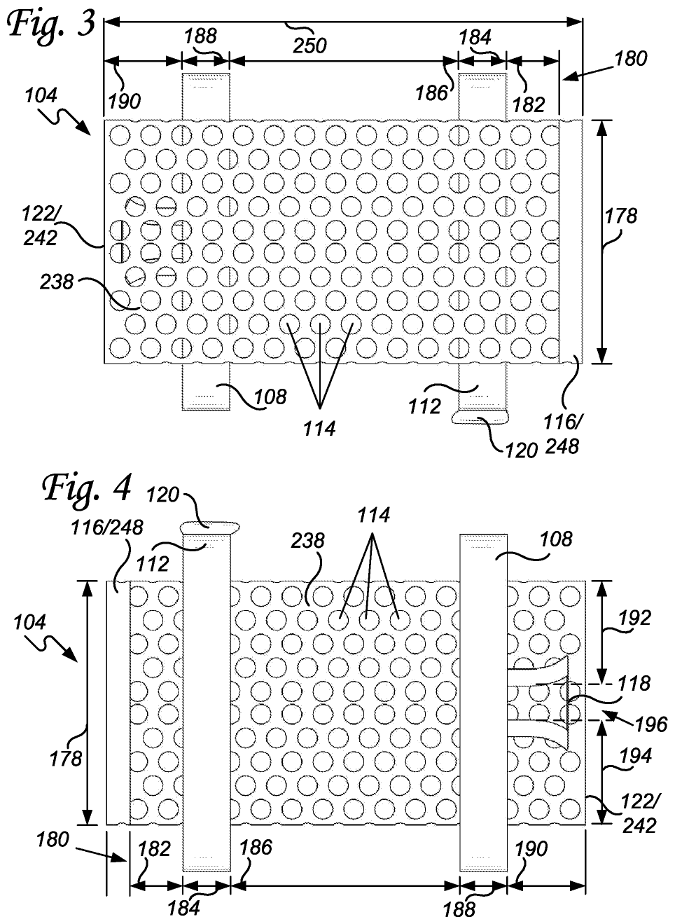

Referring to FIG. 3, there is illustrated one example of a top view of a medical infusion pump holder band 104. In an exemplary embodiment, the plurality of holes 114 is circular in shape and size to allow patient 302 to see the medical infusion pump 202 inside the receptacle 238. In this regard, the plurality of circular-holes 114 can have a maximum diameter in the range of 3 millimeters (mm) to 7 mm and is preferably in the range of 4 mm to 6 mm. In operation, the plurality of holes 114 allows the patient 302 to monitor the medical infusion pump 202 status, drug levels, light-emitting diode (LED) indicators, digitally displayed messages, and other statuses by looking through the plurality of holes 114, instead of having to remove the medical infusion pump 202 from the receptacle 238.

In an exemplary embodiment, the restraint band 108 can be fastened to the receptacle 238 proximate to the bottom end 122 in the range 190 of being located at the bottom end 122 edge to 25 mm from the bottom end 122 edge, preferably located at the bottom end 122 edge to 10 mm from the bottom end 122 edge, and ideally located at the edge of the bottom end 122. The width 188 or the restraint band 108 can be in the range of 10 mm to 30 mm, and preferably in the range of 10 mm to 15 mm.

In an exemplary embodiment, the stabilizing band 112 can be fastened proximate to the top end 116 in the range 182 of being located at the top end 116 edge to 25 mm from the top end 116 edge, preferably located at the top end edge to 10 mm from the top end 116 edge, and ideally located at the edge of the top end 116. The width 184 of the stabilizing band 112 can be in the range of 10 mm to 40 mm, and preferably in the range of 15 mm to 25 mm.

In an exemplary embodiment, distance 186 between the restraint band 108 and the stabilizing band 112 can be in the range of 80 mm to 130 mm, and preferably in the range of 85 mm to 110 mm.

In an exemplary embodiment, top seal 248 width 180 can be in the range of 2 mm to 10 mm, and preferably in the range of 3 mm to 7 mm.

In an exemplary embodiment, the size of the receptacle 238 can be selected to accommodate a wide variety of medical infusion pumps 202. Such medical infusion pumps 202 can be of different shapes and sizes. In this regard, the receptacle 238 length 250 can be in the range of 90 mm to 170 mm, and preferably in the range of 110 mm to 140 mm. The width 178 of the receptacle 238 can be in the range of 60 mm to 100 mm, and preferably in the range of 70 mm to 85 mm. The size ranges of the receptacle 238 can be the same for the medical infusion pump holder band 104, the medical infusion pump holder glove 102, and the medical infusion pump holder sleeve 106. In a plurality of exemplary embodiment, the receptacle 238 size can be adjusted in size as may be required and/or desired in a particular embodiment.

An advantage, in the present invention, is that, in an exemplary embodiment, receptacle 238 can be removable and interchangeable with the medical infusion pump holder band 104, the medical infusion pump holder glove 102, or the medical infusion pump holder sleeve 106. In a kit form, the kit can comprise one receptacle 238, one medical infusion pump holder band 104, one medical infusion pump holder glove 102, and one medical infusion pump holder sleeve 106. Other kit combinations can be selected as may be required and/or desired in other exemplary embodiments.

Referring to FIG. 4, there is illustrated one example of a bottom view of a medical infusion pump holder band 104. In an exemplary embodiment, a thumb band 118 forms a continuous loop, is stretchable and is fastened to the restraint band 108. The thumb band 118 can be fastened to the restraint band 108 at a mid-point along with the band 108 where distances 192 and 194 are equal. The attach point ends of the thumb loop 118 can be spaced a distance 196 apart in the range of coexisting at the same point to 30 mm, and preferably in the range of coexisting at the same point to 10 mm.

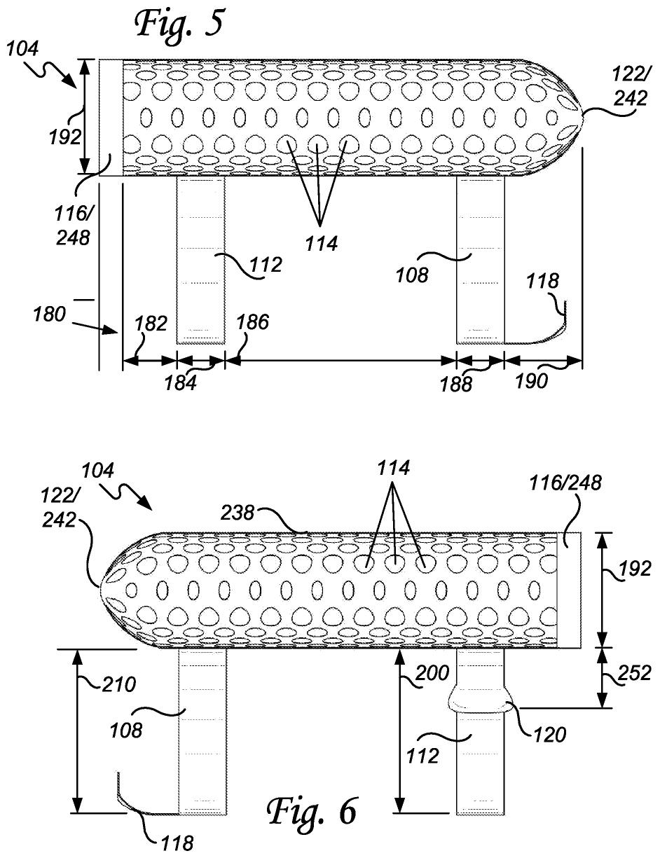

Referring to FIG. 5, there is illustrated one example of a right side view of a medical infusion pump holder band 104. In an exemplary embodiment, the top end 116 with the stretchable seal 248 is configured to be stretched open by patient 302 allowing the medical infusion pump 202 to be inserted into the receptacle 238. When released by patient 302 the stretched seal 248 constricts holding the receptacle 238 from slipping, rotating, or egressing from the receptacle 238. In an exemplary embodiment, the front end 116 can be stretched open in the diameter range of 65 mm to 120 mm, and preferably in the diameter range of 75 mm to 100 mm. In the front seal 248 constricted closed position, the front end 116 can have a diameter range of 20 mm to 60 mm, and preferably in the diameter range of 30 mm to 50 mm.

Referring to FIG. 6, there is illustrated one example of a left side view of a medical infusion pump holder band 104. In an exemplary embodiment, the resealable closure 120 is positioned at one end of the stabilizing band 112 in the range 252 of 5 mm to 70 mm from the base of the receptacle 238, and preferably in the range of 10 mm to 50 mm from the base of the receptacle 238.

In an exemplary embodiment, the diameter 210 of the stabilizing band 112 can be in the patient 302 adjustable diameter range of 60 mm to 120 mm, and preferably in the patient 302 adjustable diameter range of 60 mm to 110 mm. Ideally, the diameter of the stabilizing band 112 is selected to accommodate the size range of the patient's forearm.

In an exemplary embodiment, the diameter 200 of the restraint band 108 can be in the unstretched diameter range of 60 mm to 120 mm, and preferably in the diameter range of 60 mm to 90 mm. Ideally, the diameter of the restraint band 108 is selected to accommodate the size range of the patient's hand.

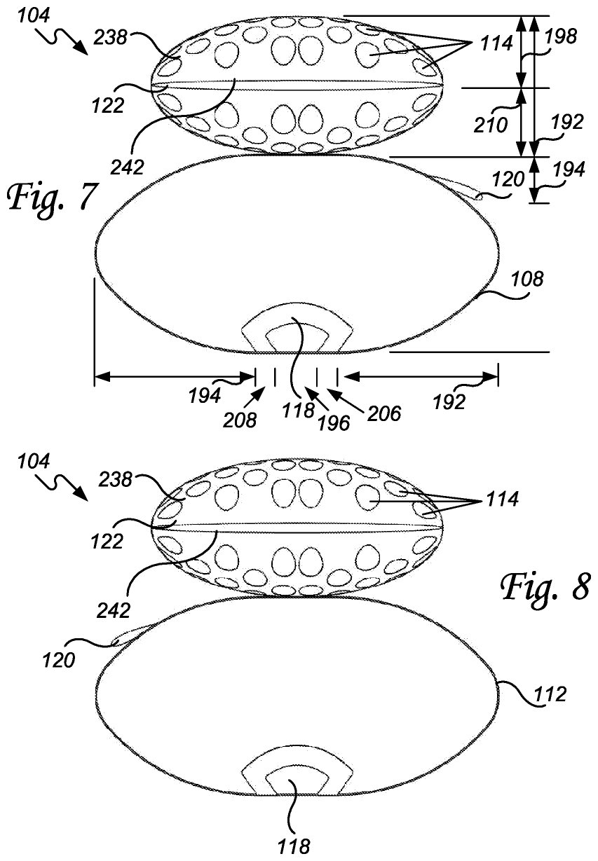

Referring to FIG. 7, there is illustrated one example of a back view of a medical infusion pump holder band 104. In an exemplary embodiment, the rear seal 242 is located equal distance along the back end 122 of the receptacle 238 where distances 198 and 210 are equal.

In an exemplary embodiment, the thumb loop 118 can be in the unstretched diameter range of 20 mm to 60 mm, and preferably in the diameter range of 25 mm to 40 mm. Ideally, the diameter of the restraint band 108 is selected to accommodate the size range of the patient's thumb. The width 206 and 208 of the thumb loop 118 can be in the range of 5 mm to 30 mm, and preferably in the range of 10 mm to 20 mm.

Referring to FIG. 8, there is illustrated one example of a front view of a medical infusion pump holder band 104.

Referring to FIG. 9, there is illustrated one example of a medical infusion pump holder glove 102. In an exemplary embodiment, a medical infusion pump holder configured as a glove 102 comprises a receptacle 238 formed from a flexible mesh material having a plurality of holes 114 dispersed throughout. The receptacle 238 has a bottom end 122 and a top end 116. A medical infusion pump 202 is inserted into receptacle 238. The receptacle 238 applies uniform constricting pressure across the surface of the medical infusion pump 202 to secure the medical infusion pump 202 from movement during motion by a patient 302 such as arm, leg, or hand motion. An intravenous drug delivery tube 204 is detachably connected to the medical infusion pump 202. Only the intravenous drug delivery tube 204 egresses from the receptacle 238. A bottom seal 242 is integrated around the periphery of the bottom end 122 and closes the bottom end 122. A top seal 248 is integrated around the periphery of the top end 116. The top seal 248 is stretchable and under a no-load condition draws the top end 116 substantially closed retaining the medical infusion pump 202 inside the receptacle 238.

A circular hand wrap 242 has a finger opening 128, a wrist opening 124, a thumb opening 126, and a top side 244. The receptacle 238 is fastened to the top side 244 fitting between the finger opening 128 and the wrist opening 124 with the bottom end 122 facing the finger opening and the top end 116 facing the wrist opening 124.

A wrist seal 254 is fastened around the periphery of the wrist opening 124, the wrist seal 254 is stretchable and under a no-load condition draws the wrist opening 124 substantially closed.

In operation, the medical infusion pump holder glove 102 is configured to receive the hand of a patient 302 in a loose-fitting manner minimizing patient sensitive skin irritation for those patients 302 who have that condition. The patient's 302 thumb slides into the thumb opening 126 preventing the medical infusion pump holder glove 102 from rotating around the hand of the patient 302. The circular hand wrap 242 substantially encloses the top portion and palm of the patient's 302 hand.

In an exemplary embodiment, the wrist seal 254 constricts around the wrist of patient 302 to prevent the medical infusion pump holder glove 102 from sliding up the forearm or down the hand of patient 302.

In an exemplary embodiment, the finger opening 128 is loose-fitting around the portion of the patient's hand where the fingers begin. The loose-fitting finger opening 128 allows patient 302 to move freely and use their hand while wearing the medical infusion pump holder glove 102 without encumbering finger movement or inducing medical infusion pump 2020 motion with finger movement.

The circular hand wrap 242 is tailored around the patient's 302 thumb knuckle allowing the patient's 302 thumb to move freely and unencumbered while the patient is wearing the medical infusion pump holder glove 102. In an exemplary embodiment, the thumb hole 126 is a concave cut in the circular hand wrap 242 material with respect to reference line 256. An advantage is that the circular hand wrap 242 is tailored around the patient thumb knuckle allowing the patient's thumb to move freely and unencumbered, and not induce medical infusion pump 202 motion with thumb movement while the patient is wearing the medical infusion pump holder glove 102.

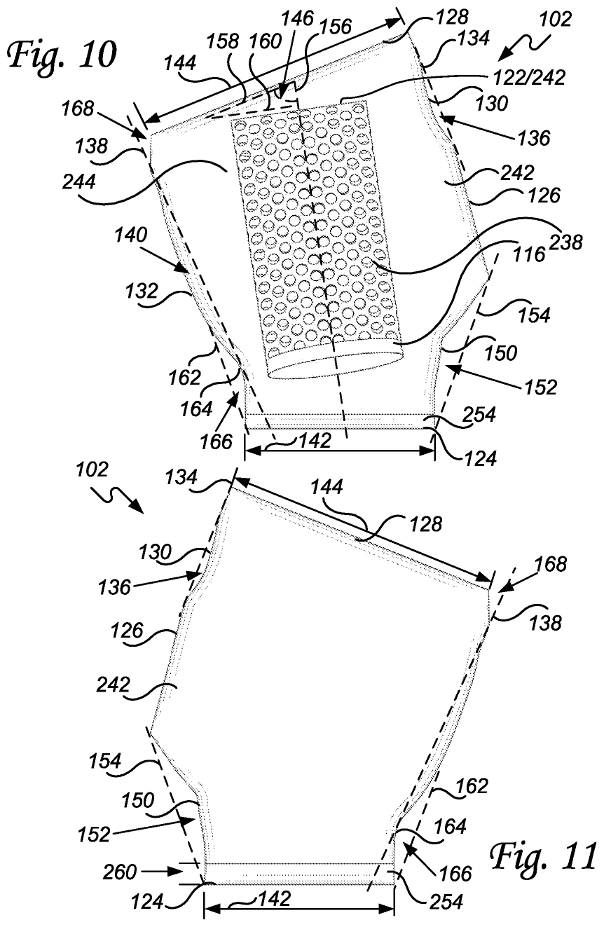

Referring to FIG. 10, there is illustrated one example of a top view of a medical infusion pump holder glove 102. In an exemplary embodiment, the front edge 130 is tailored concave 136 in the circular hand wrap 242 material with respect to reference line 134. An advantage is that the concave tailoring fits snuggly along the front side edge of the patient's hand proximate the pointer finger securing the medical infusion pump 202 from motion when patient 302 moves their hand while allowing the circular hand wrap 242 to fit loosely across the top and palm portion of the patient's 302 hand.

In an exemplary embodiment, the side portion 132 is tailor convex with respect to reference line 138. An advantage of the convex 140 tailoring of the side portion 132 is that it allows the glove 102 to fit snuggle along the side edge of the patient's hand securing the medical infusion pump 202 from motion when the patient moves their hand while allowing the circular hand wrap 242 to fit loosely across the top and palm portion of the patient's 302 hand.

In an exemplary embodiment, one end of the side edge portion 132 terminates at the finger opening 128 by being tailored inward 168 so that the circular hand wrap 242 pulls inward around the patient's pinky finger knuckle improving the medical infusion pump holder glove 102 grip along the side of the patient's 302 hand and pinky knuckle area.

In an exemplary embodiment, the medical infusion pump holder glove 102 has a left side wrist contour 164 and a right side wrist contour 150. Each of the left side wrist contour 164 and the right side wrist contour 150 can be concave 166 and 152 tailored with respect to reference lines 162 and 154 respectively to fit snuggly along the side of the patient's 302 wrist. An advantage, in the present invention, is that the medical infusion pump holder glove 102 snuggly fits along the side edges of the patient's wrist securing the medical infusion pump 202 from motion when the patient moves their hand while allowing the circular hand wrap 242 to fit loosely across the top and palm portion patient's 302 hand.

In an exemplary embodiment, the receptacle 238 is attached to the circular hand wrap 244 on the top side 244 at an angle 146 with respect to the receptacle 238 centerline reference 156, the finger opening 128 reference line 158, and the bottom of the receptacle 122 reference line 160. The receptacle is oriented with the bottom end 122 facing towards the finger opening 128 and the top end 116 facing towards the wrist opening 124.

An advantage, in the present invention, is that the receptacle 238 is mounted at the angle 146 so that the intravenous drug delivery tube 204 associated with the medical infusion pump 202 run parallel with the patient's forearm when wearing the medical infusion pump holder glove 102 so that motion of the patient's hand is unencumbered and doesn't interrupt, kink, or otherwise disrupt drug deliver at the patient 302 injection site 306. In an exemplary embodiment, the angle 146 is in the range of 50 degrees to 85 degrees, and preferably in the range of 65 degrees to 75 degrees.

In an exemplary embodiment, the width 144 of the finger opening 128 can be in the range of 90 mm to 130 mm, and preferably in the range of 100 mm to 120 mm. The width of the finger opening 128 can be selected based, in part, on the hand size of the patient 302.

A wrist seal 254 is integrated around the periphery of the wrist opening 124 and draws closed the wrist opening 124. In an exemplary embodiment, the wrist seal 254 can be an elastic band, rubber band, a drawstring, adjustable twist tie, or other types and/or kinds of seals as may be required and/or desired in a particular embodiment.

In an exemplary embodiment, the wrist opening 124 with the stretchable wrist seal 254 is configured to be stretched open by patient 302 allowing the patient's hand to slip through the wrist opening 124. When released by patient 302 the stretchable wrist 254 constricts holding the medical infusion pump holder glove 102 from slipping or rotating on the patient's 302 hand. In an exemplary embodiment, the wrist opening 124 can be stretched open in the diameter range of 90 mm to 150 mm, preferably in the diameter range of 105 mm to 125 mm. In the wrist seal 254 constricted closed position, the wrist opening 142 is in the diameter range of 40 mm to 100 mm, and preferably in the diameter range of 50 mm to 70 mm.

Referring to FIG. 11, there is illustrated one example of a bottom view of a medical infusion pump holder glove 102. In an exemplary embodiment, wrist seal 254 width 260 can be in the range of 2 mm to 10 mm, and preferably in the range of 3 mm to 7 mm.

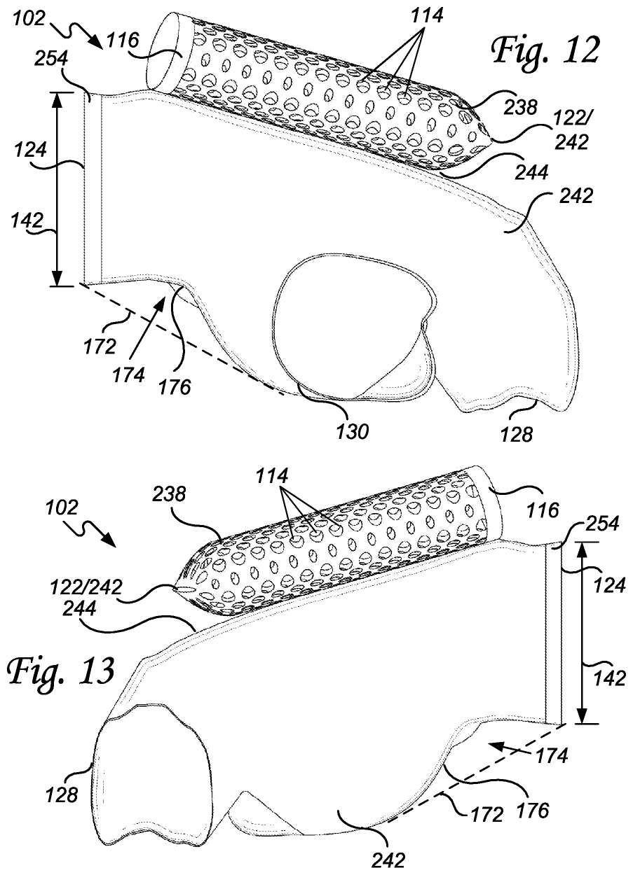

Referring to FIG. 12, there is illustrated one example of a right side view of a medical infusion pump holder glove 102. In an exemplary embodiment, the medical infusion pump holder glove 102 has a palm contour 176. The palm contour 176 can be concave 174 and tailored with respect to reference lines 172 to fit snuggly along the back edge bottom side of the patient's 302 hand proximate the wrist area. An advantage, in the present invention, is that the snug fit along the back edge bottom side of the patient's 302 hand proximate the wrist area better secures the medical infusion pump 202 from bouncing up and down on the top of the patient's 302 hand while allowing the circular hand wrap 242 to fit loosely across the top and palm portion of the patient's 302 hand.

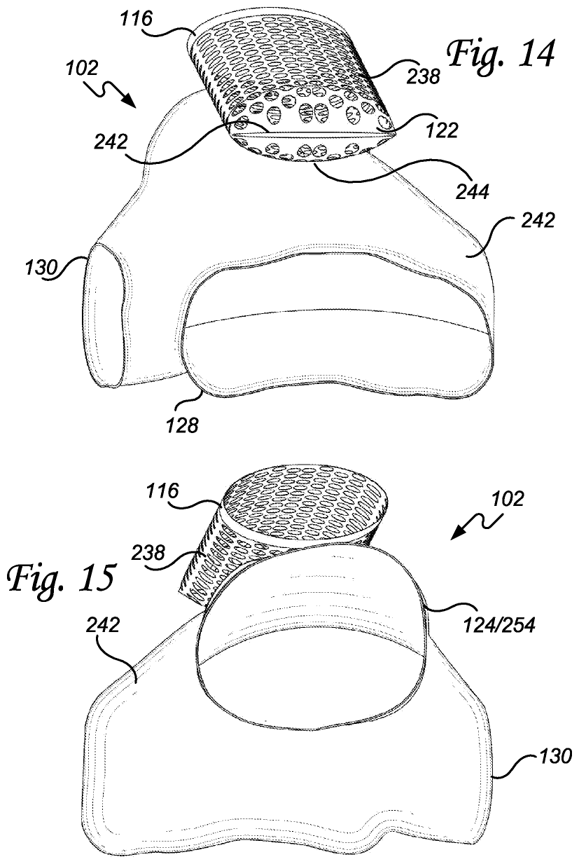

Referring to FIG. 13, there is illustrated one example of a left side view of a medical infusion pump holder glove 102. Referring to FIG. 14, there is illustrated one example of a front view of a medical infusion pump holder glove 102. Referring to FIG. 15, there is illustrated one example of a back view of a medical infusion pump holder glove 102.

Referring to FIG. 16, there is illustrated one example of a medical infusion pump holder sleeve 106 being worn on the patient's 302 arm, and FIG. 17, illustrates one example of a medical infusion pump holder sleeve 106 being worn on the patient's 302 leg.

In an exemplary embodiment, the medical infusion pump holder sleeve 106 can be worn by a patient 302 on their arm or leg proximate to an injection site 306. The medical infusion pump holder sleeve 106 comprises a receptacle 238 formed from a flexible mesh material that has a plurality of holes 114 dispersed throughout. The receptacle has a bottom end 122 and a top end 116. A medical infusion pump 202 is inserted into receptacle 238. The receptacle 238 applies uniform constricting pressure across the surface of the medical infusion pump 202 to secure the medical infusion pump 202 from movement during motion by a patient 302 such as arm, leg, or hand motion. An intravenous drug delivery tube 204 is detachably connected to the medical infusion pump 202. Only the intravenous drug delivery tube 204 egresses from the receptacle 238. A bottom seal 242 is integrated around the periphery of the bottom end 122 and closes the bottom end 122. A top seal 248 is integrated around the periphery of the top end 116. The top seal 248 is stretchable and under a no-load condition draws the top end 116 substantially closed retaining the medical infusion pump 202 inside the receptacle 238. A circular sleeve 240 formed of a compression non-slip material has a first edge 212, a second edge 214, and a top side 246. The receptacle 238 is fastened to the top side 246 fitting between the first edge 212 and the second edge 214 with the bottom end 122 facing the first edge 212 and the top end 116 facing the second edge 214. In operation, the medical infusion pump holder sleeve 206 is configured to receive the arm or the leg of the patient 302 through the central opening in the circular sleeve 240.

Referring to FIG. 18, there is illustrated one example of a top view of a medical infusion pump holder sleeve 106. In an exemplary embodiment, the first edge 212 is smaller in diameter size 218 than the second edge 214 diameter size 216 tapering 226 the shape of the circular sleeve 240 to conform with the tapered shape of the arm or the leg diameter of the patient 302 who is wearing the circular sleeve 240 creating consistent constricting pressure along the length of the circular sleeve 240 on the patient's 302 arms or leg which makes it more comfortable for the patient 302 to wear for long periods of time.

In this regard, An advantage, in the present invention, is that the medical infusion pump holder sleeve 106 can be sized for each patient 302 to create a comfortable non-slip even constricting pressure fit on their arm or leg while they wear the medical infusion pump holder sleeve 106. To this end, the diameter of a patient's arm or leg can be measured where the first edge 212 diameters 218 and the second edge 214 diameters 216 will be positioned. A medical infusion pump holder sleeve 106 can then be sized and the taper 226 determined such that the taper 226 with respect to reference lines 228 and 224 of the sleeve conforms to the taper of the patient's 302 arm or leg. A correct comfort fitting medical infusion pump holder sleeve 106 will prevent over constriction that can reduce blood flow through the extremity and generally feel uncomfortable for the patient to wear for long periods of time.

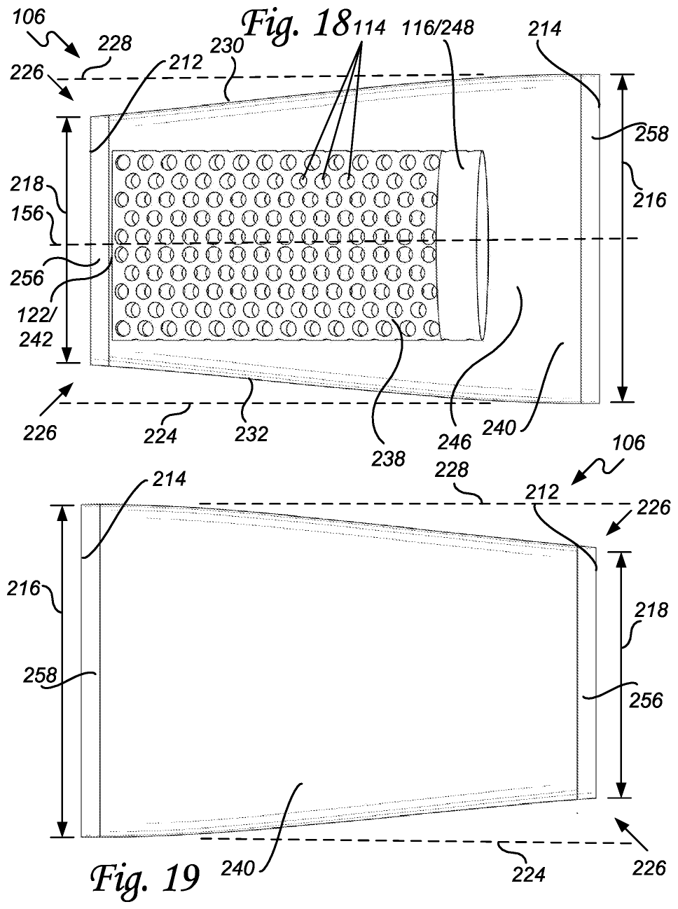

Referring to FIG. 19, there is illustrated one example of a bottom view of a medical infusion pump holder sleeve 106. In an exemplary embodiment, the first edge 212 and the second edge 214 are folded two or more times creating a first anchor edge 256 and a second anchor edge 258. The first anchor edge 256 and second anchor edge 258 increase the force required to stretch the circular sleeve 240 along the first edge 212 and the second edge 214. The increased force required to stretch the first edge 212 and the second edge 214 slightly constricts the first edge 212 and the second edge 214 into the surface of the patient's skin with respect to the rest of the circular sleeve 240 surface area. This creates anchor ridges around the circumference of the patient's arm or leg in the area of the first edge 212 and the second edge 214 that holds the medical infusion pump holder 106 from sliding or rotating on the arm or leg of the patient 302 when in motion.

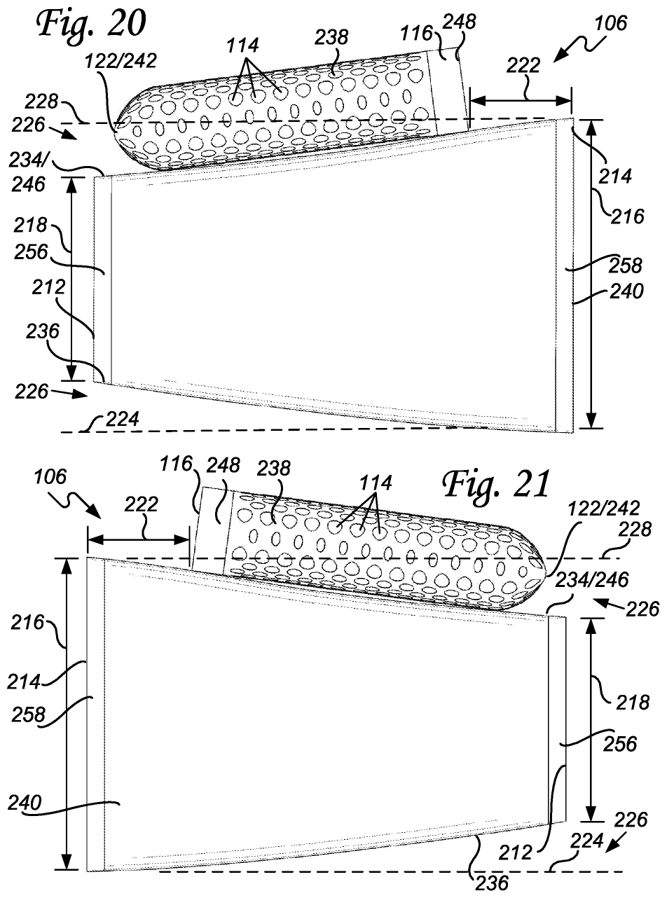

Referring to FIG. 20, there is illustrated one example of a left side view of a medical infusion pump holder sleeve 106, and FIG. 21, illustrates one example of a right side view of a medical infusion pump holder sleeve 106. In an exemplary embodiment, the bottom end 122 of the receptacle 238 is aligned with the first edge 212 creating a gap 222 between the top end 116 of the receptacle 238 and the second edge 214 such that in operation the intravenous drug delivery tube 204 at the point of connection with the medical infusion pump 202 occurs over the circular sleeve 240 avoiding contact with the skin of the patient 302.

In this regard, the intravenous drug delivery tube 204 at the point of connection with the medical infusion pump 202 can be rigid and inflexible and therefore rub at that point on the patient's 302 arm or leg. To avoid this irritation, receptacle 238 can be located with the bottom end 122 close to the first edge 212 creating a gap 222 between the top end 116 and the second edge 214 of the circular sleeve 240. This causes the intravenous drug delivery tube 204 at the point of connection with the medical infusion pump 202 to rub on the circular sleeve 240 and not the patient's 302 arm or leg.

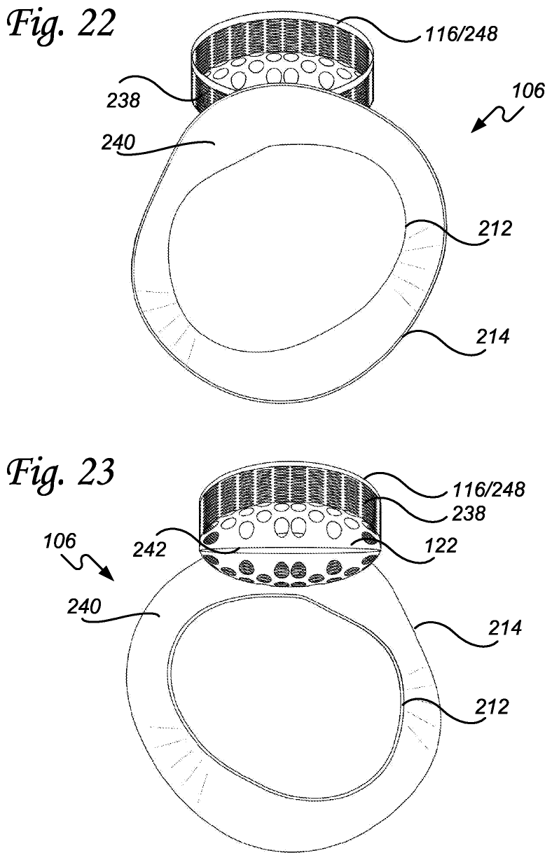

Referring to FIG. 22, there is illustrated one example of a front view of a medical infusion pump holder sleeve 106. Referring to FIG. 23, there is illustrated one example of the back view of a medical infusion pump holder sleeve 106.

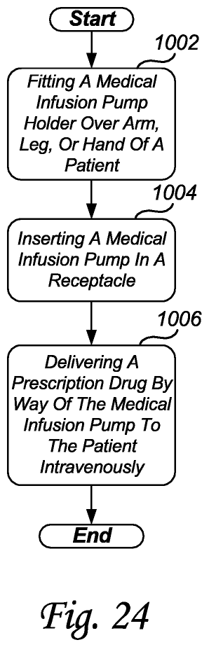

Referring to FIG. 24, there is illustrated one example of a method of using a medical infusion pump holder. In an exemplary embodiment, the method starts in step 1002 by fitting a medical infusion pump holder glove 102, band 104, or sleeve 106 over the arm, leg, or hand of the patient 302. The medical infusion pump holder sleeve 106 can be sized for each patient 302 to create a comfortable non-slip even constricting pressure fit on their arm or leg while they wear the medical infusion pump holder sleeve 106. To this end, the diameter of a patient's arm or leg can be measured where the first edge 212 diameters 218 and the second edge 214 diameters 216 will be positioned. A medical infusion pump holder sleeve 106 can then be sized and the taper 226 determined such that the taper 226 with respect to reference lines 228 and 224 of the sleeve conforms to the taper of the patient's 302 arm or leg. A correct comfort fitting medical infusion pump holder sleeve 106 will prevent over constriction that can reduce blood flow through the extremity and generally feel uncomfortable for the patient to wear for long periods of time. The method then moves to step 1004.

In step 1004, a medical infusion pump 202 is inserted in a receptacle 238. The receptacle 238 is formed from a flexible mesh material having a plurality of holes 114 dispersed throughout. The receptacle 238 has a bottom end 122 and a top end 116. A medical infusion pump 202 is inserted into receptacle 238. The receptacle 238 applies uniform constricting pressure across the surface of the medical infusion pump 202 to secure the medical infusion pump 202 from movement during motion by a patient 302 such as arm, leg, or hand motion. An intravenous drug delivery tube 204 is detachably connected to the medical infusion pump 202. Only the intravenous drug delivery tube 204 egresses from the receptacle 238. A bottom seal 242 is integrated around the periphery of the bottom end 122 and closes the bottom end 122. A top seal 248 is integrated around the periphery of the top end 116. The top seal 248 is stretchable and under a no-load condition draws the top end 116 substantially closed retaining the medical infusion pump 202 inside the receptacle 238. The method then moves to step 1006.

In step 1006, a prescription drug is delivered by way of the medical infusion pump 202 to patient 302 intravenously. The method is then exited.

The flow diagrams depicted herein are just examples. There may be many variations to these diagrams or the steps (or operations) described therein without departing from the spirit of the invention. For instance, the steps may be performed in a differing order, or steps may be added, deleted, or modified. All of these variations are considered a part of the claimed invention.

While the preferred embodiment to the invention has been described, it will be understood that those skilled in the art, both now and in the future, may make various improvements and enhancements which fall within the scope of the claims which follow. These claims should be construed to maintain the proper protection for the invention first described.

What is claimed is:

1. A medical infusion pump holder comprising:
a receptacle formed from a flexible mesh material having a plurality of holes dispersed throughout, the receptacle having a bottom end and a top end;
a medical infusion pump is inserted into the receptacle, the receptacle applies uniform constricting pressure across surface of the medical infusion pump to secure the medical infusion pump from movement during motion by a patient, an intravenous drug delivery tube is detachably connected to the medical infusion pump, only the intravenous drug delivery tube egresses from the receptacle;
a bottom seal is integrated around the periphery of the bottom end and closes the bottom end;
a top seal is fastened around the periphery of the top end, the top seal is stretchable and under no-load conditions draws the top end substantially closed retaining the medical infusion pump inside the receptacle;
a restraint band forms a continuous loop, is stretchable, and is fastened to the receptacle proximate to the bottom end;
a thumb band forms a continuous loop, is stretchable, and is fastened to the restraint band; and
a stabilizing band having a resealable closure, is non-stretchable, and is fastened proximate to the top end;
wherein the medical infusion pump holder is configured to slide over hand of the patient, the patient thumb slides into the thumb band preventing the medical infusion pump holder from rotating around hand of the patient, the restraint band wraps around hand of the patient, and the stabilizing band is tightened and secured by the patient around forearm of the patient, the medical infusion pump holder is positioned proximate to an injection site on the patient where the intravenous drug delivery tube interconnects.

2. The medical infusion pump holder in accordance with claim 1, the top seal is either an elastic band, rubber band, or a drawstring.

3. The medical infusion pump holder in accordance with claim 1, the plurality of holes are circular-shaped with a maximum diameter in the range of 3 mm to 7 mm, wherein operation and status lights on the medical infusion pump are observable through the plurality of holes.

4. The medical infusion pump holder in accordance with claim 1, the bottom seal can be opened and partially closed to allow cords from back of the medical infusion pump to egress the receptacle.

5. The medical infusion pump holder in accordance with claim 1, the stabilizing band, and the resealable closure are formed from single piece of hook-and-loop material.

6. The medical infusion pump holder in accordance with claim 1, the thumb band is fastened at mid-point of the restraint band 108.

7. The medical infusion pump holder in accordance with claim 1, the receptacle is removable and interchangeably mountable to the medical infusion pump holder when the medical infusion pump holder is configured as a band, a glove, or a sleeve.

8. A medical infusion pump holder comprising:
a receptacle formed from a flexible mesh material having a plurality of holes dispersed throughout, the receptacle having a bottom end and a top end;
a medical infusion pump is inserted into the receptacle, the receptacle applies uniform constricting pressure across surface of the medical infusion pump to secure the medical infusion pump from movement during motion by a patient, an intravenous drug delivery tube is detachably connected to the medical infusion pump, only the intravenous drug delivery tube egresses from the receptacle;
a bottom seal is integrated around the periphery of the bottom end and closes the bottom end;
a top seal is integrated around the periphery of the top end, the top seal is stretchable and under no-load conditions draws the top end substantially closed retaining the medical infusion pump inside the receptacle;
a circular hand wrap having a finger opening, a wrist opening, a thumb opening, and a top side, the receptacle is fastened to the top side fitting between the finger opening and the wrist opening with the bottom seal facing the finger opening and the top seal facing the wrist opening; and
a wrist seal is fastened around the periphery of the wrist opening, the wrist seal is stretchable and under no-load conditions draws the wrist opening substantially closed;
wherein the medical infusion pump holder is configured to receive hand of the patient in loose-fitting manner minimizing sensitive skin irritation, the patient thumb slides into the thumb opening preventing the medical infusion pump holder from rotating around hand of the patient, the circular hand wrap substantially encloses top portion and palm of the patient hand, the medical infusion pump holder is positioned proximate to an injection site on the patient where the intravenous drug delivery tube interconnects.

9. The medical infusion pump holder in accordance with claim 8, the wrist seal is either an elastic band, rubber band, or a drawstring.

10. The medical infusion pump holder in accordance with claim 8, the plurality of holes are circular-shaped with a maximum diameter in the range of 3 mm to 7 mm, wherein operation and status lights on the medical infusion pump are observable through the plurality of holes.

11. The medical infusion pump holder in accordance with claim 8, the wrist seal constricts around wrist of the patient to prevent the medical infusion pump holder from sliding up arm or down hand of the patient.

12. The medical infusion pump holder in accordance with claim 8, the finger opening is sized to be loose-fitting around hand of the patient where fingers of the patient begin.

13. The medical infusion pump holder in accordance with claim 8, the thumb opening on the top side is a concave cut in the circular hand wrap freeing thumb of the patient to move freely.

14. The medical infusion pump holder in accordance with claim 8, the receptacle is removable and interchangeably mountable to the medical infusion pump holder when the medical infusion pump holder is configured as a band, a glove, or a sleeve.

15. A medical infusion pump holder comprising:
a receptacle formed from a flexible mesh material having a plurality of holes dispersed throughout, the receptacle having a bottom end and a top end;
a medical infusion pump is inserted into the receptacle, the receptacle applies uniform constricting pressure across surface of the medical infusion pump to secure the medical infusion pump from movement during motion by a patient, an intravenous drug delivery tube is detachably connected to the medical infusion pump, only the intravenous drug delivery tube egresses from the receptacle;
a bottom seal is integrated around the periphery of the bottom end and closes the bottom end;
a top seal is integrated around the periphery of the top end, the top seal is stretchable and under no-load conditions draws the top end substantially closed retaining the medical infusion pump inside the receptacle; and
a circular sleeve formed of a compression non-slip material having a first edge, a second edge, and a top side, the receptacle is fastened to the top side fitting between the first edge and the second edge with the bottom end facing the first edge and the top end facing the second edge;
wherein the medical infusion pump holder is configured to receive arm or leg of the patient through the circular sleeve and be positioned proximate to an injection site on the patient where the intravenous drug delivery tube interconnects.

16. The medical infusion pump holder in accordance with claim 15, the first edge is smaller in diameter size than the second edge tapering shape of the circular sleeve to conform with tapered shape of arm or leg diameter of the patient wearing, creating consistent constricting pressure along length of the circular sleeve on the patient arm or leg.

17. The medical infusion pump holder in accordance with claim 15, the first edge and the second edge are folded two or more times increasing the force required to stretch which creates anchor ridges at the first edge and the second edge that constricts slightly into surface of skin of the patient with respect to rest of the circular sleeve surface area anchoring the medical infusion pump holder from sliding or rotating on arm of leg of the patient.

18. The medical infusion pump holder in accordance with claim 15, the plurality of holes are circular-shaped with a maximum diameter in the range of 3 mm to 7 mm, wherein operation and status lights on the medical infusion pump are observable through the plurality of holes.

19. The medical infusion pump holder in accordance with claim 15, the bottom end of the receptacle is aligned with the first edge creating gap between the top end of the receptacle and the second edge of the circular sleeve, wherein the intravenous drug delivery tube at point of connection with the medical infusion pump occurs over the circular sleeve avoiding contact with skin of the patient.

20. The medical infusion pump holder in accordance with claim 15, the receptacle is removable and interchangeably mountable to the medical infusion pump holder when the medical infusion pump holder is configured as a band, a glove, or a sleeve.
